(19) United States
(12) Patent Application Publication
(10) Pub. No.: US 2004/0073266 A1
(43) Pub. Date: Apr. 15, 2004

(54) Automatic detection of defibrillation lead

(76) Inventors: Paul A. Haefner, Circle Pines, MN (US); Hal M. Propp, Oakdale, MN (US); Nga Hoang Duong, Plymouth, MN (US); Gary T. Seim, Minneapolis, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/272,453

(22) Filed: Oct. 15, 2002

(51) Int. Cl. A61N 1/37

(52) U.S. Cl. 607/27

(57) ABSTRACT

An apparatus and method for appropriate selection of high energy shocking electrodes based on impedance measurements. In one example, an impedance measurement circuit measures the impedance between different sets of electrodes upon implant. The measured electrode impedance is compared to a predetermined impedance range to detect the presence of a high-energy shocking electrode. If a high-energy shocking electrode is present, a lead electrode status indicator is set. Based on the state of the lead electrode status indicator, a processor prevents or allows the use of various electrode combinations to deliver high energy therapy. Since the increase in automaticity allows the system to change the programmed therapy based on the status of the leads, patient safety is increased.

### TECHNICAL FIELD

This document relates to pacemakers, defibrillators, and any other devices that are capable of diagnosing and treating cardiac arrhythmia, and in particular, to an apparatus and method for appropriate selection of high energy shocking electrodes based on impedance measurements.

### BACKGROUND

Pacemakers deliver timed sequences of low energy electrical stimuli, called pace pulses, to the heart, such as via an intravascular lead (hereinafter referred to as a “lead”). By properly timing the delivery of pace pulses, the heart can be induced to contract in proper rhythm, greatly improving its pumping efficiency.

Defibrillators are devices capable of delivering higher energy electrical stimuli to the heart. A defibrillator is capable of delivering a high energy electrical stimulus that is sometimes referred to as a defibrillation countershock. The countershock interrupts a fibrillation, allowing the heart to reestablish a normal rhythm for efficient pumping of blood.

As these devices continue to increase in complexity, the number of leads and electrodes used by a single device increases. One problem this causes is the increased complexity in configuring the leads. Another problem is that there is an increased chance that a lead develops a poor connection or the lead becomes compromised. There is a need in the art for an improved system for detection and configuration of leads.

### SUMMARY OF THE INVENTION

This document discusses an apparatus and method for appropriate selection of high energy shocking electrodes based on impedance measurements. In one example, an impedance measurement circuit measures the impedance between different sets of electrodes upon implant. The measured electrode impedance is compared to a predetermined impedance range to detect the presence of a high-energy shocking electrode. If a high-energy shocking electrode is present, a lead electrode status indicator is set. Based on the state of the lead electrode status indicator, a processor prevents or allows the use of various electrode combinations to deliver high energy therapy. Since the increase in automaticity allows the system to change the programmed therapy based on the status of the leads, configuring the system is greatly simplified.

In a second example, impedance measurements, made during high voltage therapy delivery, are used to determine the suitability of various shocking electrodes. Alternate shocking electrodes may be chosen by a processor if the condition of the primary electrodes is compromised. If the alternate electrodes are also compromised, the processor may continue its selection process until a suitable set of electrodes is identified.

In a third example, daily impedance measurements are used to determine the suitability of various electrodes. Alternate shocking electrodes may be chosen by a processor if the condition of the primary electrodes is compromised. If the alternate electrodes are also compromised, the processor may continue its selection process until a suitable set of electrodes is identified.

This summary is intended to provide an overview of the subject matter of the present patent application. It is not intended to provide an exclusive or exhaustive explanation of the invention. The detailed description is included to provide further information about the subject matter of the present patent application.

### BRIEF DESCRIPTION OF THE DRAWINGS

In the drawings, where like numerals refer to like components throughout the several views,

FIG. 1 is a general illustration of one embodiment of portions of a system for treating cardiac arrhythmia.

FIG. 2 is a block diagram of portions of a device for treating cardiac arrhythmia coupled to a heart.

FIGS. 3 through 6 illustrate example embodiments of treating cardiac arrhythmia by disposing leads around selected cardiac regions.

FIG. 7 is a flow chart of one example of a method of a device for treating cardiac arrhythmia automatically changing the lead combination used to deliver shock therapy based on lead impedance measurements.

FIG. 8 is a flow chart of one example of a method of an external programmer changing the lead combination used to deliver shock therapy based on lead impedance measurements.

### DETAILED DESCRIPTION

In the following detailed description, reference is made to the accompanying drawings which form a part hereof, and in which is shown by way of illustration specific embodiments in which the invention may be practiced. Other embodiments may be used and structural changes may be made without departing from the scope of the present invention.

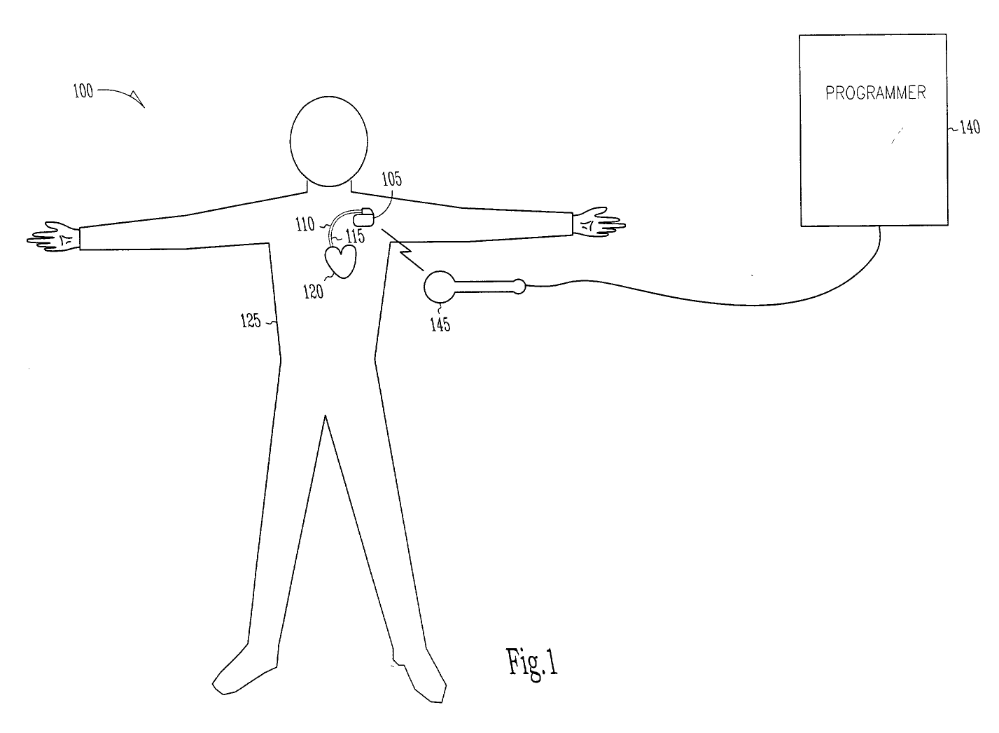

FIG. 1 shows one embodiment of portions of a system for treating cardiac arrhythmia 100. System 100 includes an implantable pulse generator (PG) 105 that is connected by a first cardiac lead 110 and a second cardiac lead 115, or one or more additional leads, to a heart 120 of a patient 125. Implantable PG 105 can take the form of a defibrillator, or a defibrillator that includes pacing capability. System 100 also includes an external programmer 140 that provides for wireless communication with the implantable PG 105 using telemetry device 145. The first cardiac lead 110 and the second cardiac lead 115 each include a proximal end and a distal end, where the distal end of the leads 110 and 115 are implanted in, or on, the heart 120 at a first cardiac region and a second cardiac region, respectively. Each lead includes one or more electrodes that allow for combinations of either unipolar and/or bipolar sensing and delivery of energy to the heart 120 for pacing, and/or defibrillation. In some embodiments, the one or more electrodes include electrodes such as sensing, pacing, and shocking electrodes.

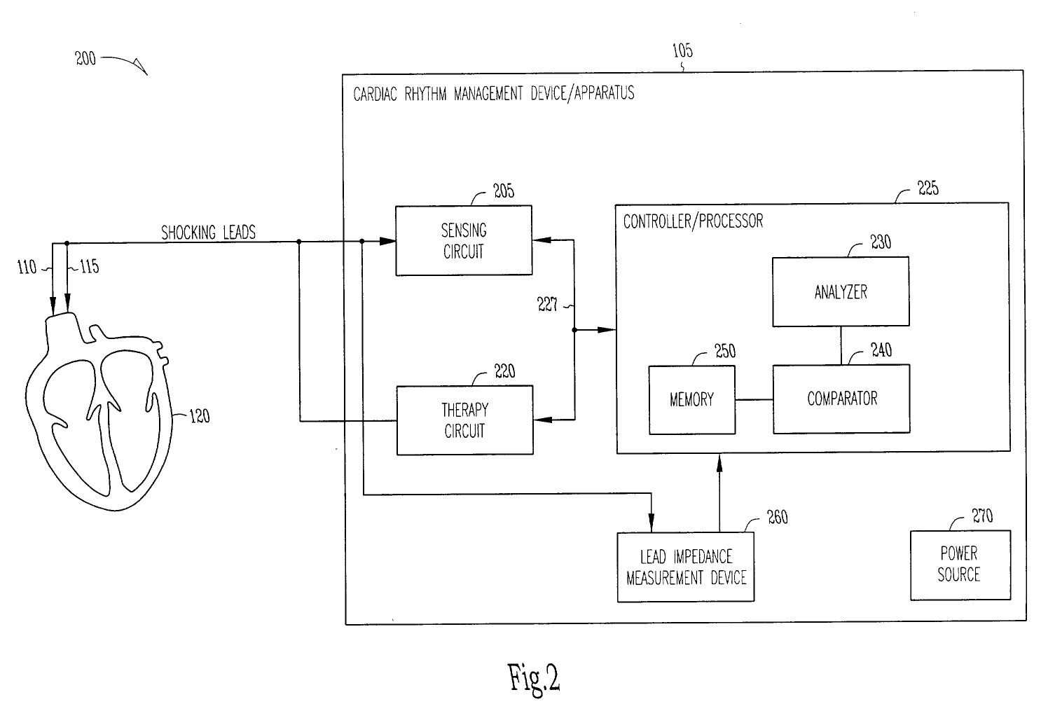

FIG. 2 is a schematic diagram of one embodiment of portions of control circuitry 200 of an implantable PG 105 coupled to the heart 120. The implantable PG 105, as shown in FIG. 2, includes a sensing circuit 205 and a therapy circuit 220 coupled to shocking leads 110 and 115. The PG 105 further includes a shocking lead impedance measurement device 260, a power source 270, and a controller/processor 225. In this embodiment, the controller/processor 225 incorporates a cardiac signal analyzer 230, a comparator 240, and a memory 250 to control device 105. In one embodiment, the functions of the analyzer 230 and the comparator 240 are implemented in software within the controller/processor 225.

Sensing circuit 205 is connected to implantable leads 110 and 115. In some embodiments, sensing circuit 205 is connected to multiple leads. Each of the leads includes one or more shocking/pacing electrodes to deliver low/high energy therapy to the heart 120. The electrodes are disposed in multiple selected cardiac regions of the heart 120, such as the coronary sinus region, the ventricular region, and the superior vena cava region. The electrodes coupled to leads 110 and 115 can include sensing, pacing, and/or shocking electrodes. Sensing circuit 205 receives cardiac signals from the sensing electrodes and amplifies the received cardiac signals.

Shocking lead impedance measurement device 260 is connected to the electrodes and measures shocking lead impedances by measuring impedance between each possible set of electrodes including at least one shocking electrode from all of the disposed electrodes. One example of a method for measuring defibrillation lead impedance is to measure the lead voltage resulting from a test current sent through the lead. This method is discussed in Linder et al. U.S. Pat. No. 6,317,628, entitled “Cardiac Rhythm Management System with Painless Lead Impedance Measurement System” and is incorporated by reference herein in its entirety, including its discussion of a lead impedance measurement of a defibrillation lead. Another example of a method for measuring defibrillation lead impedance is to calculate the impedance value from the voltage droop of a capacitively coupled output voltage pulse over a fixed period of time. This method is discussed in Citak U.S. Registered Invention No. H1,929, entitled “Cardiac Rhythm Management System with Lead Impedance Measurement” and is incorporated by reference herein in its entirety.

Each possible set of electrodes can include two or more shocking electrodes, a shocking electrode and a pacing electrode, a shocking electrode and a sensing electrode, a shocking electrode and two or more pacing/sensing electrodes, and a shocking electrode and the conductive housing that covers part of the PG 105.

In some embodiments, shocking lead impedance measurement device 260 measures shocking lead impedances between electrodes at predetermined time periods. The predetermined time period impedance measurements can include measurements performed daily or measurements performed during a programming session.

Comparator 240 which is connected to the shocking lead impedance measurement device 260, then compares each of the measured shocking lead impedances to a predetermined range of acceptable shocking lead impedance values. In one embodiment, the predetermined range of values is approximately 20 ohms to 125 ohms.

If the lead impedance measurement is within the predetermined range, analyzer circuit 230 which is connected to comparator 240 allows shock therapy to be delivered through the set of electrodes. If the measured lead impedance is outside of the predetermined range, analyzer circuit 230 prevents delivery of shock therapy using that electrode set and automatically changes the electrode set combination used to deliver the therapy.

In one embodiment, analyzer circuit 230 activates a lead electrode status indicator circuit or sets a lead electrode status indicator flag in a location in memory 250 to indicate the presence of the lead or that the integrity of the lead is not compromised. Memory 250 stores the predetermined acceptable range of shocking lead impedance values. The analyzer circuit then polls the lead electrode status indicator before delivering the therapy.

In another embodiment, processor 225 communicates the status of the lead electrodes to the external programmer 140. External programmer 140 then either allows or disallows selection of the electrode set by the programmer operator for use in shock therapy. In one example of this embodiment, when the external programmer 140 does not allow the selection of the electrode set, the external programmer 140 does not display to the programmer operator the choice of the shock therapy combination that includes the electrode set. In another embodiment, the electrode set choice is highlighted, for example by graying of the display, to indicate to the programmer operator that the choice of the electrode set is not allowed.

In another embodiment, if an impedance measurement indicates the presence of a set of electrodes, analyzer circuit 230 includes the electrode set in the periodic impedance measurements. If subsequent impedance measurements indicate that the lead has become compromised, the analyzer circuit 230 does not allow the electrode set to be used to deliver shock therapy.

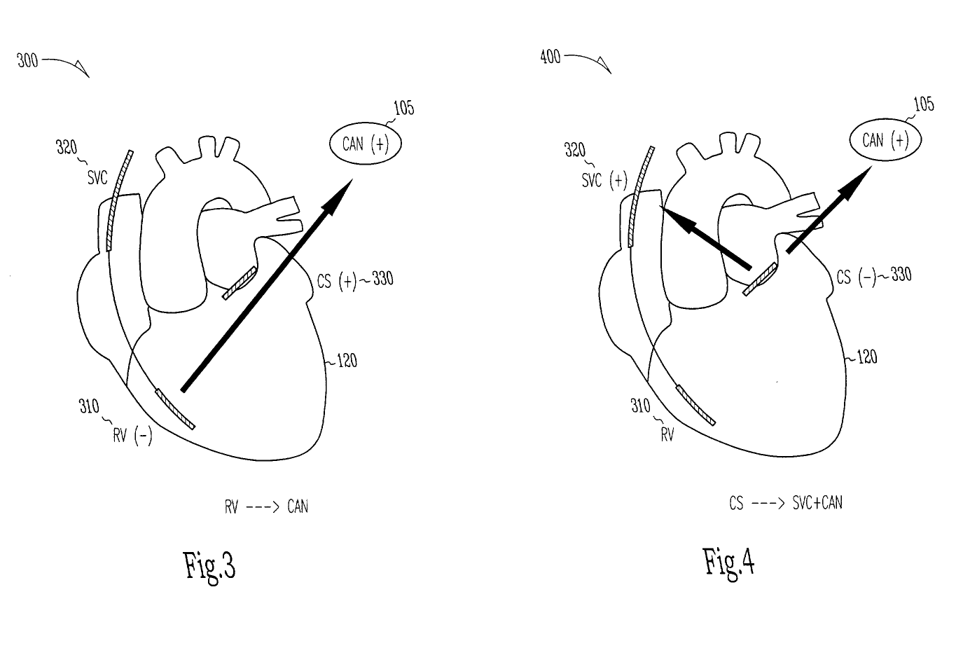

FIG. 3 illustrates one example of an embodiment 300 of treating cardiac arrhythmias by disposing leads around selected regions of the heart 120. In this embodiment leads and electrodes are shaped and sized to be disposed in the right ventricle 310 with the conductive housing covering a part of the PG 105 as the second electrode.

FIG. 4 illustrates another example of an embodiment 400 of treating cardiac arrhythmias by disposing leads around selected regions of the heart 120. In this embodiment leads and electrodes are shaped and sized to be disposed in the coronary sinus 330 and the superior vena cava 320, and the third electrode is the conductive housing covering a part of the PG 105 adapted to be an electrode.

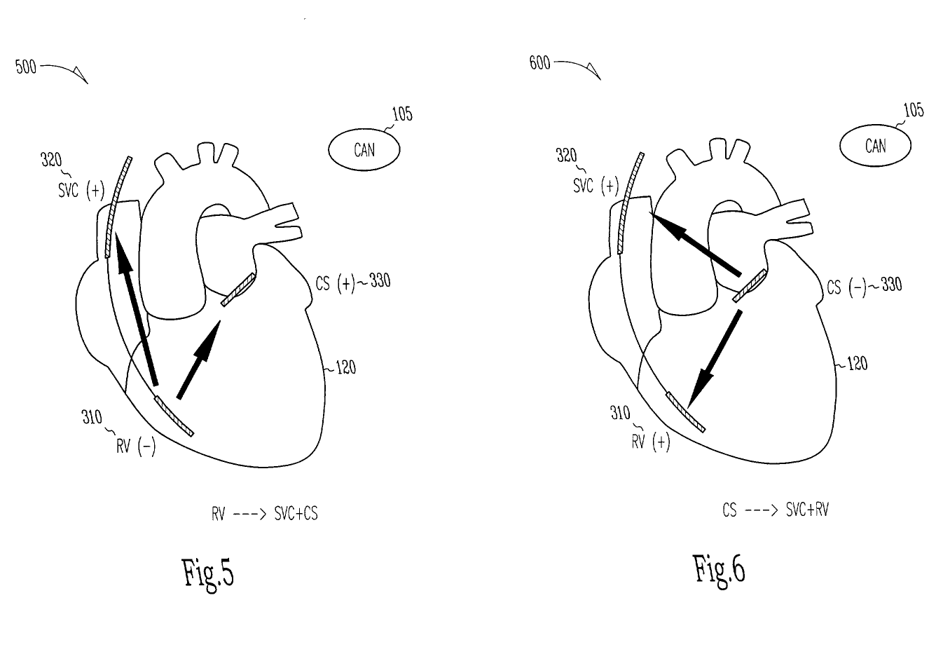

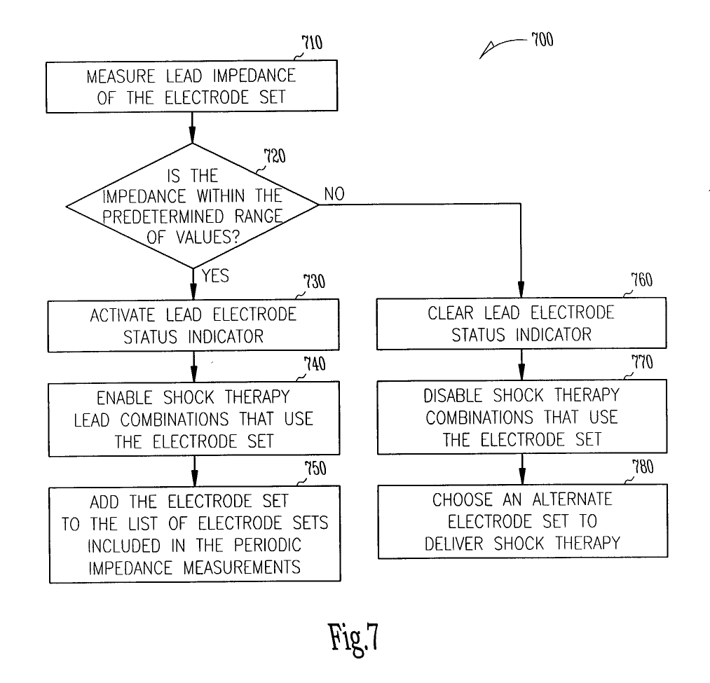

FIGS. 5 and 6 illustrate further embodiments of treating cardiac arrhythmias by disposing leads and electrodes around selected regions of the heart 120. In these embodiments leads and electrodes are shaped and sized to be disposed in the right ventricle 310, superior vena cava 320, and the coronary sinus 330. In FIG. 5, shock therapy originates from the right ventricle electrode 310. In FIG. 6, shock therapy originates from the coronary sinus 330.

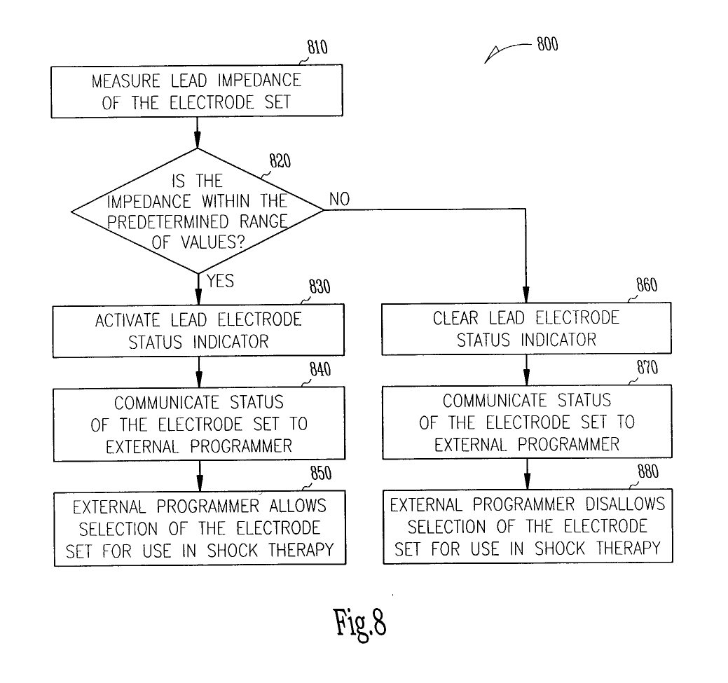

FIG. 7 is a flow chart of a method 700 of a device automatically changing the electrode set combination used to deliver shock therapy based on lead impedance measurements. At step 710 the impedance measurement is initiated. In one embodiment, the external programmer 140 initiates the impedance measurement of the electrode set. In another embodiment, the PG 105 automatically measures the impedance of the electrode set. At step 720 it is determined if the impedance measurement falls within the predetermined range. If it does fall within the range, at step 730 analyzer circuit 230 activates the lead electrode status indicator. At step 740 the analyzer enables shock therapy using that electrode set. At step 750, the analyzer adds the electrode set to the list of leads included in the periodic impedance measurements.

If the lead impedance does not fall within the range, at step 760 analyzer circuit 230 clears the lead electrode status indicator. At step 770, the analyzer circuit 230 disables shock therapy using that electrode set. At step 780, the analyzer circuit 230 chooses an alternate electrode set to deliver the shock therapy.

FIG. 8 is a flow chart of a method 800 of a device automatically changing the lead combination used to deliver shock therapy based on impedance measurements of the electrode set made during therapy delivery. At step 810 the impedance measurement is initiated. At step 820 it is determined if the lead impedance measurement falls within the predetermined range. If it does fall within the range, at step 830 analyzer circuit 230 activates the lead electrode status indicator. At step 840 the analyzer 230 communicates the status of the electrode set to the external programmer 140. At step 850, the external programmer 140 allows the programmer operator to select the electrode set, or a combination of leads that includes that electrode set, to deliver shock therapy.

If the measured impedance does not fall within the range, at step 860 analyzer circuit 230 clears the lead electrode status indicator. At step 870, the analyzer circuit 230 communicates the status of the electrode set to the external programmer 140. At step 880, the external programmer disallows the programmer operator from selecting the electrode set, or a combination of leads that includes that electrode set, to deliver the shock therapy.

In one embodiment, the analyzer 230 communicates the status of the electrode set to the external programmer 140 when the external programmer 140 interrogates the lead electrode status indicator and allows use of the electrode set if the status indication is that the electrode set is present or not compromised, and disallows use of the electrode set if the status indication is that the electrode set is not present or compromised.

Although specific examples have been illustrated and described herein, it will be appreciated by those of ordinary skill in the art that any other embodiment that exists that is calculated to achieve the same purpose may be substituted for the specific example shown. This application is intended to cover any adaptations or variations of the present invention. Therefore, it is intended that this invention be limited only by the claims and their equivalents.

In the following claims, the terms “first,” “second,” “third,” etc. are used merely as labels, and are not intended to impose numeric requirements on their objects.

What is claimed is:

1. An implantable apparatus for treating cardiac arrhythmia, the apparatus comprising:
a plurality of implantable leads and electrodes including at least one high-energy shocking electrode, wherein the leads and electrodes are shaped and sized to sense cardiac signals and deliver high and low energy therapy at multiple regions of a heart;
an impedance measurement circuit connected to the plurality of electrodes adaptable to measure the impedance between different sets of electrodes;
a comparison circuit connected to the impedance circuit, to compare the measured impedance values to a predetermined range including an upper boundary value and a lower boundary value;
an electrode status indicator circuit, wherein the electrode status indicator circuit includes a state that is active when the measured impedance between sets of electrodes is within a predetermined range; and
a processor connected to the impedance measurement circuit and the output of the electrode status indicator circuit, wherein the processor is adaptable to change the therapy delivered based on the measured impedance.

2. The apparatus of claim 1, wherein the state of the electrode status indicator is stored in a processor memory.

3. The apparatus of claim 1, wherein the leads and electrodes are shaped and sized to sense cardiac signals and deliver high and low energy therapy to multiple regions of the heart including a superior vena cava region, a coronary sinus region, and a right ventricle region.

4. The apparatus of claim 1, wherein one electrode is a conductive housing covering a part of the apparatus adaptable to be an electrode.

5. The apparatus of claim 1, wherein the processor changes the therapy delivered by changing an electrode combination used to deliver the therapy.

6. An implantable apparatus for treating cardiac arrhythmia, the apparatus comprising:
a plurality of electrical leads for sensing cardiac signals and delivering high and low energy therapy;
high energy shocking lead electrodes distributed around multiple regions of a heart, including a superior vena cava region, a coronary sinus region, and a right ventricle region;
a conductive housing covering a part of the apparatus adaptable to be an electrode;
an impedance measurement circuit connected to the plurality of leads and the conductive housing wherein the impedance circuit is adaptable to measure the impedance between sets of electrodes;
a comparison circuit connected to the impedance circuit, wherein the comparison circuit compares the measured impedance values to a predetermined range including an upper boundary value and a lower boundary value;
a lead electrode status indicator circuit, wherein the lead electrode status indicator circuit is in an active state when the measured impedance between sets of high-energy shocking electrodes is within the predetermined range; and
a processor connected to the impedance measurement circuit and the output of the lead electrode status indicator circuit, wherein the processor is adaptable to change the electrode combination that delivers the therapy.

7. The apparatus of claim 6, wherein the state of the lead electrode status indicator is stored in the processor memory.

8. The apparatus of claim 6, wherein the predetermined range of impedance values is stored in the processor memory.

9. A method comprising:
measuring an impedance between first and second lead electrodes;
comparing a value of the measured impedance to a predetermined range including an upper boundary value and a lower boundary value to detect whether a high-energy shocking lead is present; and
activating a lead electrode status indicator when the impedance is within the range indicating a presence among the first and second lead electrodes of a high-energy shocking lead electrode.

10. The method of claim 9, wherein the predetermined range is between 20 and 125 ohms.

11. The method of claim 9, wherein the presence of the high-energy shocking lead electrode initiates periodic impedance measurements for the lead.

12. The method of claim 9, wherein if the measured impedance between first and second lead electrodes is outside of the predetermined range, then selecting a third electrode to deliver high energy therapy.

13. The method of claim 12, wherein the electrode combination includes electrodes adapted for use in a right ventricle region of a heart, a superior vena cava region of the heart, and an electrode adapted from a conductive housing covering a part of an apparatus.

14. The method of claim 12, wherein the electrodes include electrodes adapted for use in a coronary sinus region of a heart, a superior vena cava region of the heart, and an electrode adapted from a conductive housing covering a part of an apparatus.

15. The method of claim 12, wherein the electrodes include electrodes adapted for use in a coronary sinus region, a superior vena region, and a right ventricular region of a heart.

16. The method of claim 12, wherein if the measured impedance of the electrode set that includes the third electrode is also outside of the predetermined range, continuing a selection process until a suitable set of electrodes is identified.

17. A system for treating cardiac arrhythmia comprising:
an external programmer; and
an implantable apparatus for treating cardiac arrhythmia, the implantable apparatus capable of determining status of a high-energy shocking electrode by an impedance measurement;
wherein the implantable apparatus communicates the status of an electrode set to the external programmer and the external programmer allows or disallows selection of a therapy combination involving that electrode set depending on the status of the electrode set.

18. The system of claim 17, wherein the implantable apparatus contains a lead electrode status indicator and communicates the status of a high impedance lead to an external programmer when the external programmer polls the lead electrode status indicator.

19. The system of claim 17, wherein disallowing selection of a therapy combination includes not displaying the therapy combination to a programmer operator.

20. The system of claim 17, wherein disallowing selection of a therapy combination includes highlighting the therapy combination in a programmer display.
